(19) United States
(12) Patent Application Publication
(10) Pub. No.: US 2021/0121097 A1
(43) Pub. Date: Apr. 29, 2021

(54) URETHRAL MEASUREMENT CATHETER

(71) Applicant: Spinal Singularity, Inc., San Clemente, CA (US)

(72) Inventors: Derek Herrera, San Clemente, CA (US); Jeffery S. Kasalko, San Clemente, CA (US)

(73) Assignee: Spinal Singularity, Inc., San Clemente, CA (US)

(21) Appl. No.: 17/080,828

(22) Filed: Oct. 26, 2020

Related U.S. Application Data
(60) Provisional application No. 62/926,326, filed on Oct. 25, 2019.

(51) Int. Cl. A61B 5/107; A61B 5/20; A61B 5/00

(52) U.S. Cl. CPC A61B 5/1076; A61M 25/0074; A61B 5/6852; A61B 5/20

(57) ABSTRACT

Described is a urinary measurement catheter (UMC), which has a tube, a distal tip, a bulbar urethral anchor, and a bladder anchor, and that can be used to measure anatomical profiles of the urethra. Using the UMC according to one method comprising the following steps can be used to measure the entire urethral length: (a) insert the UMC through the urethra until the bladder anchor is positioned in the bladder; (b) pulling back on the UMC until the bladder anchor is positioned against the bladder neck; (c) mark or clamp the UMC at the meatus, or otherwise identifying the location of the UMC tube at the meatus; and (d) remove the UMC from the body and measure the distance from the location of the UMC tube as determined in step (c) at the meatus to a distal edge of the bladder anchor.

### CROSS-REFERENCE TO RELATED APPLICATIONS

This application claims priority to U.S. Provisional Application No. 62/926,326 entitled Urethral Measurement Catheter, which was filed on Oct. 25, 2019, the contents of which are incorporated herein by reference.

### FIELD

This disclosure relates to devices, systems and methods that can be used to determine the dimensions/characteristics of the urethra, and that can also empty the bladder.

### BACKGROUND

Many people have varying anatomical profiles and different dimensions of the length, width, and shape of their urethra. The different sizes may be a result of age, genetics, size, and/or other factors.

Currently, methods used to measure the length, width, and profile of the urethra include cystoscopy (using flexible or rigid endoscopes), ultrasound, and other methods.

These dimensions can be valuable for clinicians who are treating patients for a variety of conditions including Benign Prostate Hyperplasia (BPH), Urinary Retention, Prostate Cancer, and other conditions.

Some patients utilize a Foley Indwelling Catheter to empty urine from their bladder. These catheters may remain positioned in the body up to 30 days and utilize a balloon in the bladder to retain them in place.

The Foley Indwelling Catheter is relatively simple to use. One major drawback to its design is that the balloon must be inflated inside of the bladder. If the balloon is inflated anywhere in the urethra there is a chance for serious damage and trauma to the urethra that may result in significant injury to the patient.

Such trauma may be difficult to treat and require urethrotomy or other invasive techniques. In some cases, this incident has led and/or contributed to patient death.

Another key challenge with the Foley Indwelling Catheter is that once the balloon is inflated any forces on the tube may result in trauma to the bladder neck. If the collection bag or exposed tubing is caught on anything then the catheter may be ripped from the body causing significant trauma to the patient.

Another similar trauma that patients experience is with individuals experiencing cognitive decline or other neurological disorders like Alzheimer's, Dementia, etc. Such patients may forget why they have a catheter positioned in the bladder, or become agitated and try to pull the catheter out resulting in similar trauma as stated above.

Over time, the prolonged use of Foley Indwelling Catheters can lead to urethral erosion and tissue damage. This is a problem and may require invasive surgical techniques to rebuild the tissue. The goal of these surgeries is to enable the patient to regain some level of continence, but often these surgeries themselves have serious challenges that threaten the wellbeing of the patient.

The following are incorporated herein by reference: U.S. application Ser. No. 15/072,345 to Herrera et al., entitled Extended-Use Valved Urinary Catheter, and filed on Mar. 16, 2016; PCT Application Serial No. PCT/US2016/014648, entitled Bladder Management Systems, and filed on Jan. 23, 2016; U.S. application Ser. No. 15/545,903 to Herrera et al., entitled Bladder Management System, and filed on Jul. 27, 2017; U.S. application Ser. No. 15/721,096 to Herrera et al., entitled Urinary Prosthesis Systems, and filed on Sep. 29, 2017; U.S. Pat. No. 9,775,698, entitled Urinary Prosthesis Systems; U.S. application Ser. No. 15/785,403 to Herrera, entitled Extended-Use Catheters, and filed on Oct. 16, 2017; U.S. application Ser. No. 15/785,405 to Derek Herrera entitled Catheter Mating Devices, and filed on Oct. 16, 2017; U.S. application Ser. No. 15/785,398 to Derek Herrera entitled Catheters and Catheter Mating Devices and Systems, and filed on Oct. 16, 2017.

### SUMMARY

A such urethral measuring catheter (“UMC”) having a tube, a distal tip, a bulbar urethral anchor, and a bladder anchor, can be configured and used to measure lengths and widths inside of the body in which the UMC is positioned, such as in a bladder, urinary tract, and urethra. One exemplary method according to the disclosure for measuring total urethral length comprises the steps of: (a) inserting the UMC through the urethra until the bladder anchor is positioned in the bladder; (b) pulling back on the UMC until the bladder anchor is positioned against the bladder neck; (c) marking or clamping the UMC at the meatus, or otherwise identifying the location of the UMC tube at the meatus; and (d) removing the UMC from the body and measuring the distance from the location of the UMC tube as determined in step (c) at the meatus to a distal edge of the bladder anchor.

Another exemplary method according to the disclosure for measuring the width and/or length of a portion of the body, comprises the steps of: (a) inserting a UMC through the urethra until the second anchor is positioned in the bladder; (b) moving fluid into a flexible tube until an outer wall of the flexible tube expands to be in contact with a surface of the body orifice in which the tube is positioned; and (c) measuring the width of the flexible tube when it is in contact with the surface of the body orifice.

The respective anchors may be configured to allow for varying distances between them to accommodate varying prostatic urethral lengths and external urinary sphincter lengths. The total UMC length may be sized to accommodate the patient's anatomy.

### BRIEF DESCRIPTION OF THE DRAWINGS

FIG. 1 is a side view of a urethral measurement catheter (UMC) according to this disclosure.

FIG. 2 is an enlarged side view of a UMC according to this disclosure.

FIG. 3 is a cross-sectional view of a multi-lumen UMC according to this disclosure.

FIG. 4 illustrates how to measure total urethral length.

FIG. 5 compares the shape of a bladder anchor of the present disclosure to that of a Foley balloon catheter anchor.

FIG. 6 shows in cross section the male urethra and bladder and female urethra and bladder.

FIG. 7 is a side view of the UMC of FIG. 1 with an extendable flap 19 in its non-extended position.

FIG. 8 is a rear side, partial, perspective view of the UMC of FIG. 7.

FIG. 9 is an end, cross-sectional view of the UMC of FIG. 8 taken through line D-D.

FIG. 10 is a partial, side, perspective view of part of the UMC of FIGS. 8 and 9.

FIG. 11 is a partial side view of a UMC with a dual extendable flap 190 in its extended position.

FIG. 12 is a side, end, partial, perspective view of the UMC of FIG. 11.

FIG. 13 is a partial, side, cross-sectional view of the UMC of FIG. 12.

FIG. 14 is a view of part of the UMC of FIG. 12 taken through line H-H.

FIG. 15 is a side view of an exemplary embodiment of a UMC retainer portion according to aspects of the invention.

FIG. 16 is a cross-sectional view of the retainer portion of FIG. 15 taken along lines 3A-3A.

FIG. 17 is a cross-sectional view of the outer wall of the retainer portion taken along line 3A-3A.

FIG. 18 is a cross-sectional view of the expansion structure of the retainer portion shown in FIG. 16 with the expansion structure in its expanded position.

### DETAILED DESCRIPTION

The present disclosure relates to devices, systems and methods that may use, include or be a urethral measurement catheter (“UMC”) 10 designed to measure various lengths and widths of the urethra.

In some embodiments, the UMC 10 may comprise a first anchor 19 or 290 in the bladder (also called one or more expandable flaps 19, or dual expandable flaps 290) and a second anchor 20 in the bulbar urethra (also called a retainer or bulbar urethra anchor). Each of the anchors 19 or 290 and 20 may have any appropriate shape, size, material, and material properties to enable a suitable force for insertion, removal, and to allow the UMC 10 to be retained in the bladder once positioned correctly. The distances between the two anchors 19 (or 290) and 20 may also enable a clinician to estimate the lengths of portions of the urethra, including the prostatic urethral length, external urinary sphincter length, total urethral length, and/or combinations of these lengths.

Each anchor 19 (or 290) and 20 can be compressed to fit inside the adult male urethra, and to expand in portions of the urethra that are wider and that permit the anchor to expand. One example is the bulbar urethra, which is wider than the pendulous urethra and external urinary sphincter. Thus, an anchor that has been moved through the pendulous urethra expands in the bulbar urethra. In this disclosure, the bladder anchor 19 or 290 is positioned in the bladder and the bulbar urethra anchor 20 is retained in the bulbar urethra when the UMC 10 is properly positioned in the body.

### Retainer Portion or Bulbar Urethra Anchor

The retainer portion 20 is positioned in the bulbar urethra when UMC 10 is properly positioned in the lower urinary tract of a human male. Retainer portion 20 is configured to prevent the inadvertent migration of UMC 10 either forward or backward once UMC 10 is properly positioned in the body. If positioned in the bulbar urethra, the retainer portion 20 is blocked by the external sphincter to prevent inadvertent retrograde migration, and blocked by the penile portion of the urethra to prevent inadvertent ante grade migration. When sufficient pulling or pushing force is applied to UMC 10, retainer portion 20 compresses from its second, expanded position to its first, compressed position so that it can pass through the penile portion of the penile portion of the urethra. In this manner, UMC 10 can be removed from, or being placed in, the lower urinary tract.

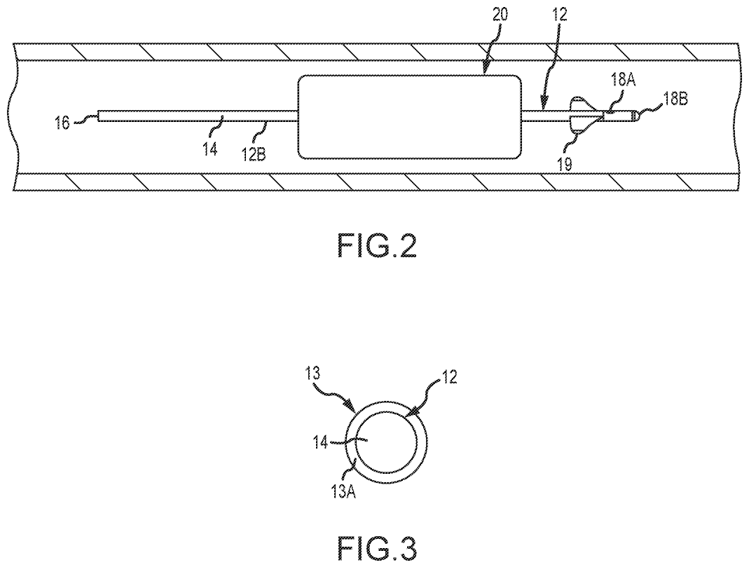

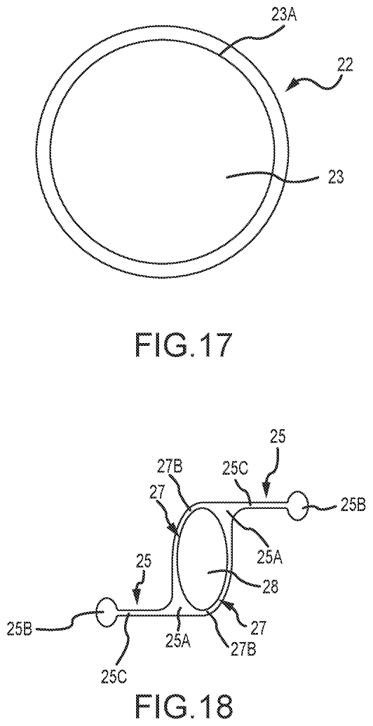

Retainer portion 20 is positioned between the distal end 18B and the proximal end 16. Turning to FIGS. 2, 7, and 15-17, in the embodiment shown, retainer portion 20 has (1) an outer wall 22 that has an outer surface 22A, (2) an inner surface 22B, (3) a first, compressed position in which it is configured to fit through the penile urethra, and (4) a second, expanded position in which it has a maximum cross-sectional area measured around the outer surface 22A that is at least 30% greater than the first cross-sectional area of UMC tube 12. When in the second, expanded position, retainer portion 20 is configured so that it cannot fit through the penile urethra. The retainer portion 20 is configured to have a maximum diameter of 0.3 mm to 8.0 mm when in the first, compressed position, and is configured to have a maximum diameter of 4.0 mm to 20 mm when in the second, expanded position. Retainer portion 20 has a cavity 23 inside of the outer wall 22.

An internal structure inside of the cavity 23 includes a core 27 and at least one expansion structure 25. The core 27 has a passage 28, in which the tube 12 is positioned, and a wall 27B. As shown, the at least one expansion structure 25 is connected to wall 27B of core 27, extends therefrom, and is configured to contact inner surface 22B of the outer wall 22 and exert outward force on the outer wall 22 to urge it towards its second, expanded position.

In the embodiment shown, the internal structure comprising core 27 and at least one expansion structure 25 is a single piece made in any suitable manner, such as by molding, over-molding, or extruding. In this embodiment, the inner structure is not formed with outer wall 22, but is formed separately and outer wall 22 is positioned over it. For example, tube 12 can be positioned inside of passage 28 of core 27, and then outer cover 22 can be positioned over tube 12 and expansion structure 25.

The retainer portion 20 as shown comprises two expansion structures 25 in the cavity 23 and each expansion structure 25 is configured to apply outward force on the outer cover 22. As shown, each expansion structure 25 is wing shaped and extends outward from the core 27. Each expansion structure 25 has a first end 25A connected to, or integrally formed with, the core 27 and a second end 25B that terminates in an enlarged sphere 25C. Each expansion structure 25 has an intermediate section 28D that touches inner wall 22B of the outer cover 22 in order to apply outward pressure.

Each expansion structure 25 has a length (as measured from first end 25A to second end 25B) that is at least 30%, at least 40%, or at least 50% of the circumference of the inner wall 25B of the outer cover 22, or has a length that is greater than 50% of the circumference of the inner wall 22B of the outer cover 22, such as at least 60%, or at least 70%, or at least 80% of the circumference.

Each expansion structure 25 has an intermediate section 25D having a thickness that is 25% or more, or 30% or more, or 40% or more of the thickness of the wall of outer cover 22 (i.e., the thickness as measured between outer wall 22A and inner wall 22B).

The outer wall 22 of the retainer portion 20 can be physically compressed to ½ or less of the maximum cross-sectional area when subjected to a compressive force evenly applied along the outer wall 22 of an amount from: 1-5 lbs., or 2-4 lbs., or 2-6 lbs., or 4-6 lbs., or 5-10 lbs., or 7-10 lbs., or 5-22 lbs.

Or, the outer wall 22 can be compressed from the second, expanded position to the first, compressed position, when the outer wall 22 is subjected to a compressive force evenly applied along the outer wall 22 of an amount from: 1-5 lbs., or 2-4 lbs., or 2-6 lbs., or 4-6 lbs., or 5-10 lbs., or 7-10 lbs., or 5-22 lbs.

Outer cover 22 of retainer portion 20 can comprise ribs, dimples, staples, or other structures on its outer surface to help retain it in the bulbar urethra or other body area.

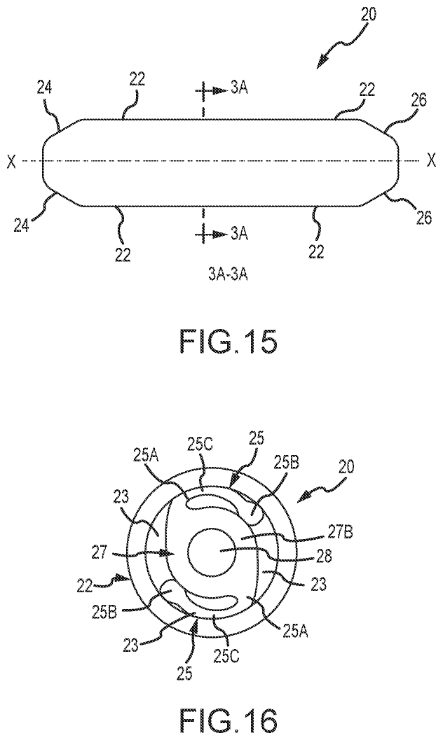

As best seen in FIGS. 15-17, the retainer portion 20 can comprise an outer surface 22A, proximal tapered surface 24, and a distal tapered surface 26. The proximal tapered surface 24 can be tapered from the outer surface 22A to about the outer surface 12B of tube 12. The distal tapered surface 26 can be tapered from the outer surface 22A to about the outer surface 12A of tube 12. The retainer portion has a length as measured along the longitudinal axis X. In one embodiment the retainer portion 20 has a total length of any amount from about: 1 cm to 10 cm, or 2 cm to 8 cm, or 3 cm to 7 cm, or 4 cm to 6 cm, and top surface 22 has a length of any amount from about: 1 cm to 10 cm, or about 10%, about 20%, about 30%, or about 40%, or about 50%, or about 60%, or about 70%, or about 80%, or about 90%, or about 95%, or any amount from about 5% to 95%, of the total length of retainer portion 20.

In one exemplary embodiment, the overall length of retainer portion 20 is about 40 mm, the overall uncompressed width at its center is about 22 mm, the thickness of wall 25C is about 0.75 mm, or any thickness between about 0.70 and 0.90 mm, or about 0.75 to 0.85 mm, and the radius of end 25B is about 2.0 mm, or any amount from about 0.5 mm and 2.1 mm.

In an embodiment suitable for use in the lower urinary tract of a human male, the maximum cross-sectional area as measured inside of outer surface 22A (and including the cross-sectional area of passage 28) is: (a) greater than the cross-sectional area of the external sphincter, (b) greater than the cross-sectional area of the penile urethra, and (c) smaller than the cross-sectional dimension of the bulbar urethra. The maximum cross-sectional area (as measured when retainer portion 20 is in its second, expanded position) may be 1.2 times larger, 1.5 to two times larger, three times as large, four times as large, five times as large, six times as large, seven times as large, eight times as large, nine times as large, ten times as large, or any amount from: 1.2 to five times as large, or 1.5 to ten times as large, as the cross-sectional area measured inside the outer surface 12B of tube 12. The maximum cross-sectional area (as measured when retainer portion 20 is not being compressed) may be any amount from: (24 mm)²π to (25 mm)²π, (4 mm)²π to (25 mm)²π, or (6 mm)²π to (20 mm)²π, or (8 mm)²π to (16 mm)²π, or (10 mm)²π to (15 mm)²π, or (12 mm)²π to (15 mm)²π, or (5 mm)²π to (10 mm)²π. In one embodiment the outer surface 22A has a circular cross-sectional shape and has a diameter of any amount from: 5 mm to 10 mm, or 5 mm to 7 mm, or 4 mm to 8 mm, or 6 mm to 15 mm, or 8 mm to 15 mm, or 6 mm to 20 mm, or 8 mm to 22 mm. The diameter of surface 12B (which is the outer diameter of tube 12) can be about 2.0 mm to 6.0 mm, or 4.6 to 6.0 mm, or any amount from: 1.5 mm to 6.5 mm.

### Bladder Anchors

The bladder anchor (e.g., the one or more extendable flaps 19 and/or dual extendable flaps 290) may also be designed in a manner that improves post-void residual (PVR) volume. For example, in the embodiment shown, the anchor comprises one or more extendable flaps 19 and/or dual extendable flaps 290, rather than a spherical balloon, so the bladder outlet is not blocked as compared to Foley Indwelling Catheter balloons.

Both anchors 19, 20 and/or 290 may be designed with material properties to facilitate insertion/removal by utilizing an appropriate insertion force and removal force, while maintaining appropriate retention forces. The anchors may include suitable symmetric or asymmetric shapes, sizes, varying durometers and/or materials, and be made using any suitable manufacturing method (such as over molding or injection molding).

When a UMC 10 according to this disclosure is removed from the body, it can be pulled out gently by using sufficient removal force without causing unnecessary trauma to the urethra. This is for both purposeful and accidental removals. It is not necessary to use a syringe or other device to remove the UMC. Only appropriate removal pressure using one's hand is required. The preferred measure force for removal of the UMC 10 is about 0.5-5 Newtons, or 1-5 Newtons, or 0.5-2 Newtons, or 1-3 Newtons, or 1-4 Newtons, or any amount between 0.5 Newtons and 5 Newtons.

### Extendable Flap Anchor 19

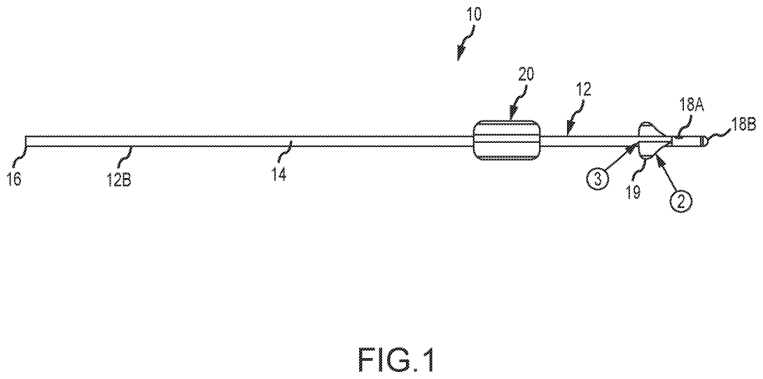

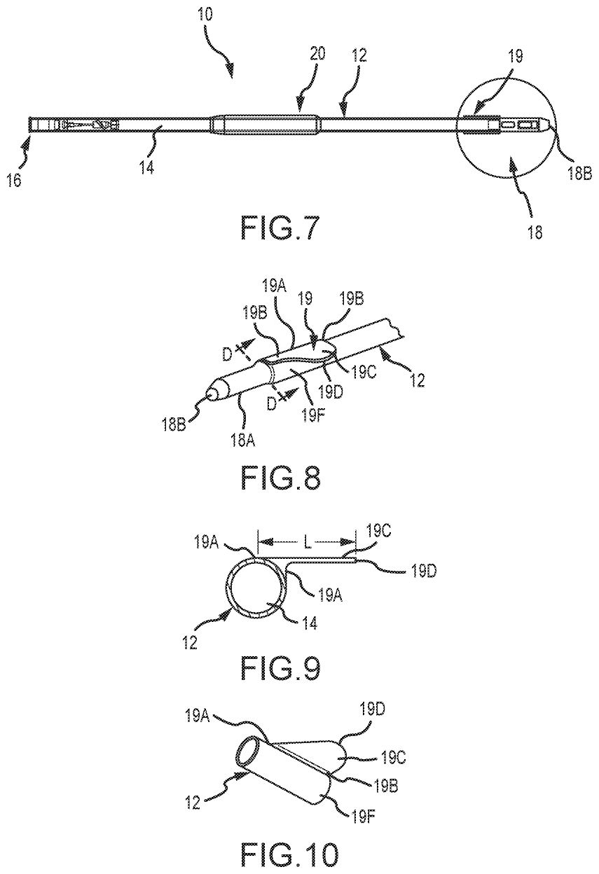

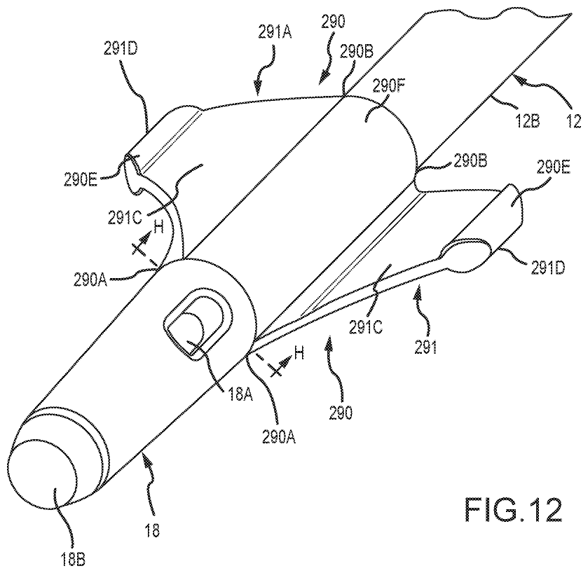

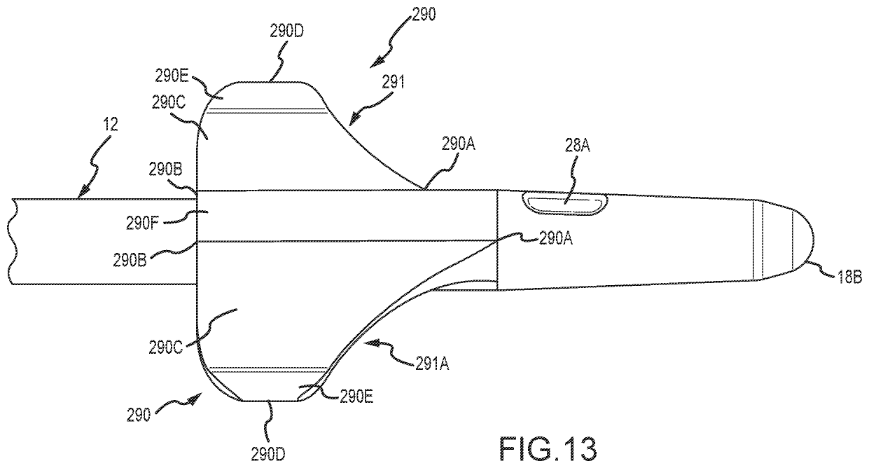

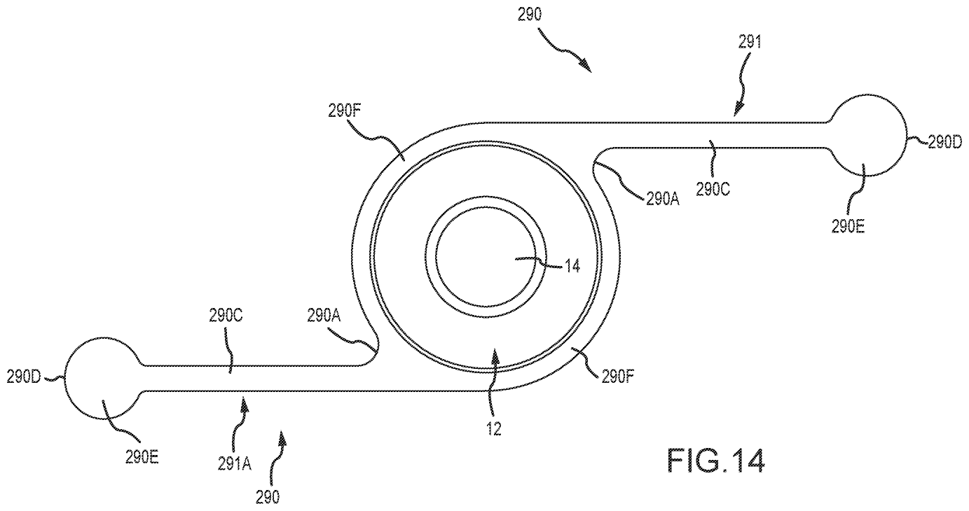

The UMC may comprise an extendable flap 19 on the distal end 18, as shown in FIGS. 1B-1E. The extendable flap 19 has a first, contracted position in which it is positioned against the outer surface 12B of tube 12, and a second, extended position (shown in FIGS. 7 to 14) in which it extends outward from the outer surface 12B of tube 12. The extendable flap 19 may be comprised of silicone or other suitable, medical grade material(s).

The extendable flap 19 preferably has a length (as measured from the position where it connects to outer wall 12B of tube 12 to the outermost edge 19D) that is at least 20%, or at least 30%, or at least 40%, or at least 50%, or at least 60%, or at least 70%, or at least 80%, or at least 90%, or at least 100%, or at least 120%, or at least 150%, or any amount from 20% to 150% of the outer circumference of the tube 12, as measured around outer surface 12B. The extendable flap 19 preferably has a thickness that is at least 10%, at least 20%, at least 30%, at least 40%, at least 50%, at least 60%, at least 70%, at least 80%, at least 90%, at least 100%, at least 110%, at least 120%, at least 130%, at least 1540%, at least 150%, or any amount from 10% to 150% of the thickness of the tube wall, as measured from outer surface 12B to inner surface 12A.

The extendable flap 19 may be of suitable thickness, length, and hardness suitable for use of the UMC. For example, the flap could utilize a 70A shore hardness with a 0.6 mm wall thickness as measured along length L from one edge of the flap 19 to the other as shown in FIG. 9. Flap 19 has an axial length as measured from 19A to 19B that is preferably about equal to the length, or any amount from about 50% of the length to about 200% of the length, such as at least 10%, at least 20%, at least 30%, at least 40%, at least 50%, at least 60%, at least 70%, at least 80%, at least 90%, at least 100%, at least 110%, at least 120%, at least 130%, at least 1540%, at least 150% at least 160%, at least 170%, at least 180%, at least 190%, or at least 200%.

The extendable flap 19 may comprise a thicker portion, or thicker ridge along each edge 19A as compared to the thickness of the rest of dual extendable flap 19. For example, the thickness at that location could be thicker than the thickness measured at the center of portion 190C by any amount from 10% to 100%, such as 10%, 20%, 30%, 40%, 50%, 60%, 70%, 80%, 90%, or 100%. This added thickness at that location may aid the user in collapsing the dual extendable flap 19 for insertion and also provide additional rigidity to support proper anchoring.

Extendable flap 19 can be integrally formed with the tube 12 or formed separately and attached to tube 12. As shown in this embodiment, extendable flap 19 is a separate component that is pulled over tube 12, and it has a body portion 19F.

### Dual Extendable Flap Anchor

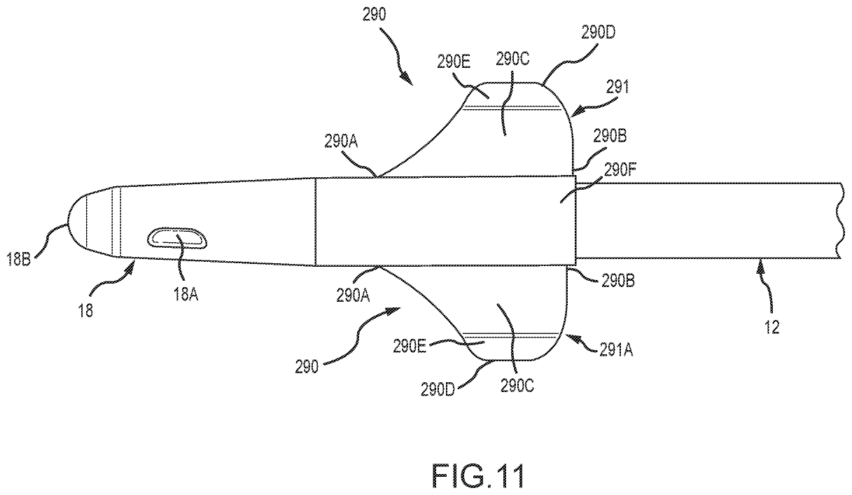

The UMC may comprise a dual extendable flap 290 on the distal end 18, as shown in FIGS. 11-14. The dual extendable flap 290 as shown has two, identical flaps 291, 291A positioned 180 degrees from one another on tube 12. Flaps 291, 291A may, however, be positioned at any suitable location and need not be sized or shaped the same. The dual extendable flap 190 may be comprised of silicone or other suitable, medical grade material(s).

Each flap 291, 291A has a first, contracted position in which it is positioned against the outer surface 12B of tube 12, and a second, extended position (shown in FIGS. 11-14) in which flaps 291, 291A extend outward from the outer surface 12B of tube 12.

Each flap 291, 291A of the dual extendable flap 290 preferably has a length (as measured from the position at which it connects to outer wall 12B of tube 12 to the outermost edge 290D) that is at least 20%, or at least 30%, or at least 40%, or at least 50%, or at least 60%, or at least 70%, or at least 80%, or at least 90%, or at least 100%, or at least 120%, or at least 150%, or any amount from 20% to 150% of the outer circumference of the tube 12, as measured around outer surface 12B. Each flap 291, 291A preferably has a thickness at portion 290C that is at least 10%, at least 20%, at least 30%, at least 40%, at least 50%, at least 60%, at least 70%, at least 80%, at least 90%, at least 100%, at least 110%, at least 120%, at least 130%, at least 1540%, at least 150%, or any amount from 10% to 150% of the thickness of the tube wall, as measured from outer surface 12B to inner surface 12A.

Each flap 291, 291A may be of suitable thickness, length, and hardness suitable for use of the UMC 10. For example, the dual extendable flap 290 could utilize a 70 A shore hardness with a 0.6 mm wall thickness as measured along length L in section 190C. Each flap 291, 291A has an axial length as measured from 290A to 290B that is preferably about equal to the length, or any amount from about 50% of the length to about 200% of the length, such as at least 10%, at least 20%, at least 30%, at least 40%, at least 50%, at least 60%, at least 70%, at least 80%, at least 90%, at least 100%, at least 110%, at least 120%, at least 130%, at least 1540%, at least 150% at least 160%, at least 170%, at least 180%, at least 190%, or at least 200%.

The dual extendable flap 290 may comprise a thicker portion, or thicker ridge along each edge 290A as compared to the thickness of the rest of dual extendable flap 290. For example, the thickness at that location could be ticker than the thickness measured at the center of portion 290C by any amount from 10% to 100%, such as 10%, 20%, 30%, 40%, 50%, 60%, 70%, 80%, 90%, or 100%. This added thickness at that location may aid the user in collapsing the dual extendable flap 290 for insertion and also provide additional rigidity to support proper anchoring.

Further, each extendable flap 291, 291A as should has a bulb 290E at its respective tips. The bulb 290E as shown is circular in cross section and has a diameter that is greater than the thickness of section 290C. For example, each bulb 290E could be have a diameter that is any amount from 10% to 200% greater than the thickness of section 290C, such as being greater by at least: 10%, 20%, 30%, 40%, 50%, 60%, 70%, 80%, 90%, 100%, 100%, 120%, 130%, 140%, 150%, 160%, 170%, 180%, 190%, or 200%.

Dual extendable flap 290 can be integrally formed with the tube 12 or formed separately and attached to tube 12. As shown in this embodiment, dual extendable flap 190 is a separate component that is pulled over tube 12, and it has a body portion 190F.

### Use

The anchors may also be designed such that the anchor in the bladder (e.g., the one or more extendable flaps 19 and/or dual extendable flaps 290) can pass through the external urinary sphincter and the anchor in the bulbar urethra (e.g., the retainer 20) is configured not to pass through the external urinary sphincter. This design may enable clinicians to identify the point at which the anchor in the bulbar urethra and/or the anchor in the bladder is at or near the external urinary sphincter.

The distance from the anchor 20 in the bulbar urethra to the proximal end 16 of the UMC 10 is long enough so the proximal end extends beyond the tip of the penis, and preferably at least 1″-4″ beyond the tip, so that the proximal end 16 of the UMC 10 is outside OF the body when the distal end 18B of the UMC 12 and the bladder anchor 19 or 290 is properly positioned in the bladder. The UMC 10 may also have an appropriate shape (similar to a Foley Indwelling Catheter) at the proximal end 16 (i.e., the end that is positioned outside of the penis tip) that allows a patient to secure the proximal end 16 of the UMC 10 to the body.

There is also preferably no valve in the tube lumen 14 so the UMC 10 has an unrestricted lumen that allows for continuous drainage of urine once opening(s) 18A at the distal end 18B of the UMC 10 that open into the lumen 14 are inside of the bladder. Clinicians may then advance the UMC 10 an additional 2-3 cm to ensure the bladder anchor (e.g., the one or more extendable flaps 19 and/or dual extendable flaps 290) are seated inside the bladder. The bladder anchor 19 or 290 provides appropriate removal force to retain the device in the bladder during use.

The UMC 10 may be designed to allow for extended use of up to thirty days in the body.

### Measuring Widths

Utilizing the UMC 12, the width of the urethra could be measured by using different sized bladder anchors (e.g., the one or more extendable flaps 19 and/or dual extendable flaps 290), which would function as a go/no-go gauge through various orifices. Another method of measuring widths, or diameters, utilizing the UMC 10 is to include an additional tube (or tubes) comprised of soft material that run the length of the UMC 10. In one embodiment shown in FIG. 3, the additional one or more tubes 13 would surround the tube 12 of the UMC 10, and define an inner lumen 13A that is outside of the UMC 10 tube wall 12B. Fluid could be injected into the inner lumen 13A and the soft material and would be visible under fluoroscopy. For example, fluid could be injected (preferably at a known pressure) to expand the soft tube wall 13 to the diameter of the orifice, such as the urethra, in which the UMC 10 is positioned. Then the fluid in lumen 13A could be imaged and measured to determine the width of the urethra or other orifice.

### Measuring Lengths

A UMC 10 may be used in a procedure that permits clinicians to obtain measurements for the total urethral length, prostatic urethra length, and bladder neck to external urinary sphincter length.

To obtain the total urethral length the clinician may gently pull back on the UMC 10 once the distal end 18 and bladder anchor 19 or 290 are inserted in the bladder to confirm the bladder anchor 19, 290 is seated at the bladder neck. The clinician may then mark/clamp or otherwise identify the location of the tube 12 at the meatus. When the UMC 10 is removed from the body, the measurement for total urethral length may be obtained by measuring the distance from the mark on tube 12 where the meatus was identified to the distal edge of the bladder anchor 19 or 290. Another way to determine the total urethral length is to measure from the mark of the meatus on tube 12 (made as described above) to the distal tip 18B of the UMC and subtract the distance from the distal tip 18B to the distal-most part of the bladder anchor 19 or 290. Another way to obtain the total urethral length is to measure from the mark of the meatus on tube 12 (made as described above) to the distal tip 18B of the UMC 10, subtract that amount from the total overall length of the UMC tube 12, and then subtract the distance of the distal UMC tip 18B to the distal edge of the bladder anchor 19 or 290.

To obtain the measurement for bladder neck to external urinary sphincter, a clinician may push the UMC 10 forward and determine the position at which resistance is detected from the first anchor (e.g., the retainer 20) pressing against the external urinary sphincter. The clinician would then pull back to where resistance is felt by the bladder anchor 19 or 290 against the bladder sphincter. This distance can then be subtracted from the distance between the two anchors 20 and 19 or 290 to obtain the measurement.

The prostatic urethra length may be measured by utilizing the distance between the two anchors 20 and 19 or 290. The clinician may insert the UMC 10 and confirm the bladder anchor 19 or 290 is properly seated inside the bladder at the bladder neck. This would be Position X. Then the clinician can apply force to push the UMC 10 deeper into the bladder and identify how far the UMC can be inserted before resistance is felt when the urethral anchor 20 reaches the external urinary sphincter. This would be Position Y. The clinician may subtract the distance between Position X and Position Y from the distance between the urethral anchor 20 and the bladder anchor 19 or 20 to obtain the prostatic urethra length plus the external urinary sphincter length. Known estimates for the external urinary sphincter length range from 0.5-1.5 cm so this distance may be subtracted from the distance between the urethral anchor 20 and the bladder anchor 19 or 290 to obtain an estimate of the prostatic urethra length.

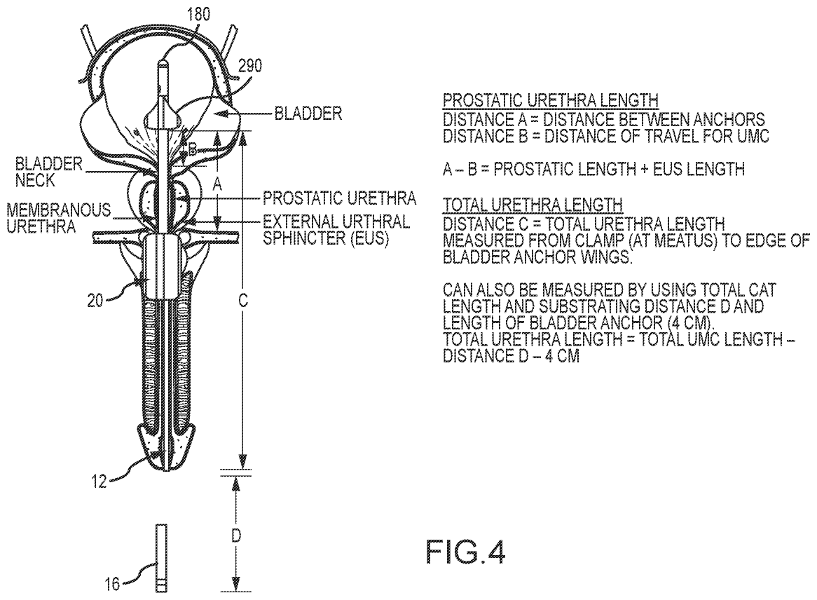

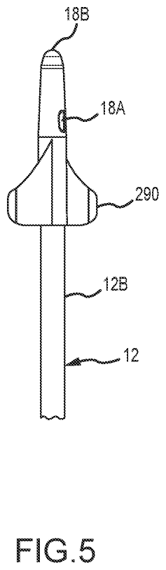

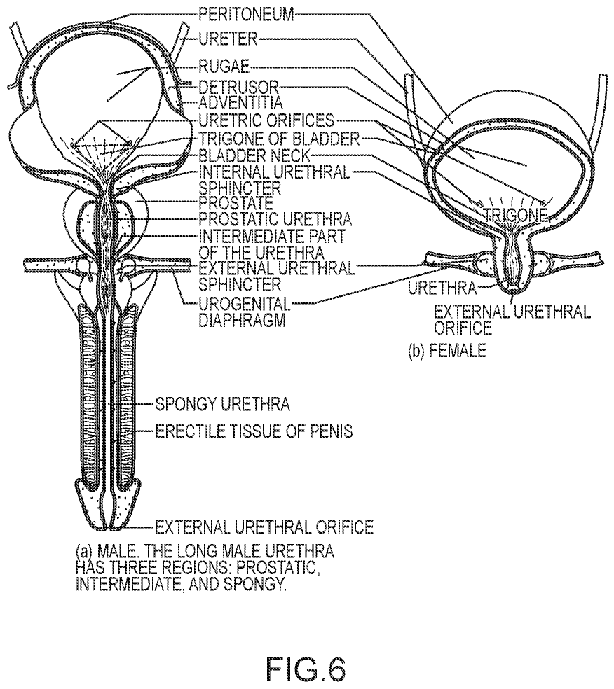

Turning to FIGS. 4 and 6, the external urinary sphincter is a portion of the urethra that is just distal to the prostatic portion of the urethra. The distance that the UMC 10 is measuring in the instance discussed above is equal to the distance of the prostatic urethra+the distance of the external urinary sphincter. One example of this would be prostatic urethra length=4.5 cm, and external urinary sphincter length=1.0 cm

In this instance, the measurement the UMC 10 would provide using the steps outlined in this application would be 5.5 cm. So in this application “prostatic urethra length plus external urinary sphincter length” means the addition of the lengths of each segment of the urethra.

In one embodiment, gradations and markings using extrusion molding techniques or pad printing, or any suitable method, can be visible/tactile on the outer surface of the UMC 10 tube 12 to facilitate length measurements. The markings/gradations may follow a simple numbering scheme (0-40 cm) and/or may also include colors to aid in visual identification and to lessen error in measurement.

The UMC 10 may also serve similar functions for the female anatomy and be sized to account for the varying anatomical differences.

### Anchoring Outside of the Body

In some embodiments, the proximal end 16 of the UMC 10 may have a shape that is similar to the Foley Indwelling Catheter near the proximal tip. Such a design may permit the UMC 10 to be anchored to the outside of the body using standard adhesive mounting methods that are similar to Foley Indwelling Catheters. This assists with securement of the UMC 10 and helps to minimize any accidental or unnecessary removals.

### SOME NON-LIMITING EXEMPLARY EMBODIMENTS OF THIS DISCLOSURE

### Example 1

A method for measuring total urethral length utilizing a urethral measuring catheter (“UMC”) having a tube, a distal tip, a bulbar urethral anchor, and a bladder anchor, the method comprising the steps of:
(a) inserting the UMC through the urethra until the bladder anchor is positioned in the bladder;
(b) pulling back on the UMC until the bladder anchor is positioned against the bladder neck;
(c) marking or clamping the UMC at the meatus, or otherwise identifying the location of the UMC tube at the meatus; and
(d) removing the UMC from the body and measuring the distance from the location of the UMC tube as determined in step (c) at the meatus to a distal edge of the bladder anchor.

### Example 2

A method for measuring the total urethral length utilizing a urethral measuring catheter (“UMC”) having a tube, a distal tip, a bulbar urethral anchor, and a bladder anchor, the method comprising the steps of:
(a) inserting the UMC through the urethra until the bladder anchor is positioned in the bladder;
(b) pulling back on the UMC until the bladder anchor is positioned against the bladder neck;
(c) marking or clamping the UMC at the meatus, or otherwise identifying the location of the UMC tube at the meatus; and
(d) removing the UMC from the body and measuring the distance from the location of the UMC tube as determined in step (c) at the meatus to the distal end of the UMC.

### Example 3

A method for measuring the total urethral length utilizing a urethral measuring catheter (“UMC”) having a tube, a distal tip, a bulbar urethral anchor, and a bladder anchor, the method comprising the steps of:
(a) inserting the UMC through the urethra until the bladder anchor is positioned in the bladder;
(b) pulling back on the UMC until the bladder anchor is positioned against the bladder neck;
(c) marking or clamping the UMC at the meatus, or otherwise identifying the location of the UMC tube at the meatus; and
(d) removing the UMC from the body and measuring the distance from the location of the UMC tube as determined in step (c) at the meatus to a distal tip of the UMC, and subtracting from that the distance from the distal UMC tip to the distal edge of the UMC.

### Example 4

The method of example 1 that further comprises the step of obtaining the measurement for the bladder neck to the external urinary sphincter by pushing forward on the UMC until resistance is felt from the urethral anchor pressing against the external urinary sphincter, measuring that position on the UMC, then pulling the UMC back to where resistance is felt by the bladder anchor pressing against the bladder sphincter, and subtracting that distance from the distance between the urethral anchor and the bladder anchor to obtain the measurement.

### Example 5

A method for obtaining the measurement for the bladder neck to external urinary sphincter, utilizing a urethral measuring catheter (“UMC”) having a tube, a urethral anchor and a bladder anchor, the method comprising the steps of:
(a) inserting the UMC through the urethra until the second anchor is positioned in the bladder;
(b) pushing forward on the UMC until resistance is felt from the urethral anchor pressing against the external urinary sphincter and marking or determining that position on the UMC;
(c) pulling back on the UMC until the second anchor is positioned against the bladder sphincter and marking or clamping the UMC at the meatus, or otherwise identifying the location of the UMC tube at the meatus; and
(d) subtracting the distance between the markings in (b) and (c) from the distance between the urethral anchor and the bladder anchor.

### Example 6

A method for measuring the width of a portion of the body that utilizes an expandable tube, the method comprising the steps of:
(a) inserting a UMC through the urethra until the second anchor is positioned in the bladder;
(b) moving fluid into the flexible tube until an outer wall of the flexible tube expands to be in contact with a surface of the body orifice; and
(c) measuring the width of the flexible tube when it is in contact with the surface of the body orifice.

### Example 7

The method of example 6, wherein the flexible tube is external to a tube of a UMC.

### Example 8

The method of example 6, wherein the flexible is external to and coaxial with a tube of a UMC.

### Example 9

The method of any of examples 1-8, wherein the bladder anchor is one or more wings.

### Example 10

The method of any of examples 1-9, wherein the urethral anchor is a retainer.

### Example 11

The method of any of examples 1-10, wherein the UMC further includes gradations or markings.

### Example 12

A urethral measuring catheter (“UMC”), comprising (a) a tube, (b) a first end, (c) a second end, (d) a retainer portion between the distal end and the proximal end, the retainer portion having (i) an outer wall that has an outer surface, an inner surface, a first, compressed position in which it is configured to fit through the penile urethra, and a second, expanded position in which it has a maximum cross-sectional area at least twice as great as the first cross-sectional area and is configured to not fit through the penile urethra, (ii) a cavity inside of the outer wall, (iii) a core inside of the cavity, the core having a passage in which the tube is positioned, and (iv) at least one expansion structure positioned inside of the cavity, the at least one expansion structure being connected to the core and being configured to contact an inner surface of the outer wall and exert outward force on the outer wall, retainer portion is configured to fit in the penile urethra, and a second dimension in which the second compressible anchor is too large to fit in the penile urethra.

### Example 13

The UMC of example 12 that further includes gradations or markings.

### Example 14

The UMC of example 13 that is formed using extrusion molding.

### Example 15

The UMC of example 13 that is formed using pad printing.

### Example 16

The UMC of example 13, wherein the graduations/markings are on the outer surface of the UMC tube and are colored.

### Example 17

The UMC of example 13 or 16, wherein the graduations are tactile.

### Example 18

The UMC of example 12, wherein the tube further comprises (i) a wall with an outer surface, the outer surface having a first cross-sectional area, (ii) a lumen, (iii) a distal end with one or more openings in communication with the lumen, and (iv) a proximal end with an opening in communication with the lumen.

### Example 19

The UMC of example 12 or 18 that further comprises a valve that is operated to be in (i) a closed configuration, wherein fluid cannot flow out of the proximal end, or (ii) an open configuration in which fluid can flow out of the proximal end; and

### Example 20

The UMC of any of examples 12-19, wherein the retainer portion has a maximum cross-sectional area that is 2-3 times greater than the first cross-sectional area.

### Example 21

The UMC of any of examples 12-20, wherein the retainer portion has a maximum cross-sectional area that is 1.5-4 times larger than the first cross-sectional area.

### Example 22

The UMC of any of examples 12-21, wherein the maximum cross-sectional area is an area from: (24 mm)2π to (25 mm)2π, or (4 mm)2π to (25 mm)2π.

### Example 23

The UMC of any of examples 12-22, wherein the retainer portion is circular in cross-section at its maximum cross-sectional area, and has a diameter of 5 mm to 10 mm at the position of the maximum cross-sectional area.

### Example 24

The UMC of any of examples 12-23, wherein the retainer portion has a hardness of an amount from: 1 to 70 Shore A, or 5-15 Shore A, or 10-20 Shore A, or 5-15 Shore A, or 10-15 Shore A.

### Example 25

The UMC of any of examples 12-24, wherein the retainer portion is comprised of silicone.

### Example 26

The UMC of any of examples 12-25, wherein the outer wall of the retainer portion can be physically compressed to ½ or less of the maximum cross-sectional area when subjected to a compressive force evenly applied along the outer wall of an amount from: 3-5 lbs., or 2-4 lbs., or 1-6 lbs., or 4-6 lbs., or 5-10 lbs., or 7-10 lbs., or 5-22 lbs.

### Example 27

The UMC of any of examples 12-26, wherein the outer wall can be compressed from the second, expanded position to the first, compressed position, when the outer wall is subjected to a compressive force evenly applied along the outer wall of an amount from: 3-5 lbs., or 2-4 lbs., or 1-6 lbs., or 4-6 lbs., or 5-10 lbs., or 7-10 lbs., or 5-22 lbs.

### Example 28

The UMC of any of examples 12-27, wherein the retainer portion is configured to have a maximum diameter of 0.3 mm to 8.0 mm when in the first, compressed position, and configured to have a maximum diameter of 4.0 mm to 15 mm when in the second, expanded position.

### Example 29

The UMC of any of examples 12-27 that further includes one or more sensors on or in the UMC.

### Example 30

The UMC of example 29 that is configured such that the one or more sensors are positioned in a bladder when the UMC is positioned in a lower urinary tract of a human male.

### Example 31

The UMC of example 29 or 30, wherein the one or more sensors are positioned at least partially in the lumen.

### Example 32

The UMC of any of examples 29-31, wherein the one or more sensors are configured to collect data of the patient, the data comprising one or more of: fluid pressure inside of the bladder, fluid volume inside of the bladder, temperature inside of the bladder, acidity of urine, bacteria level and type in urine, chemical composition of urine, motion of the patient, location of the patient, and fluid flow when emptying the bladder.

### Example 33

The UMC of any of examples 12-32, wherein that includes a second lumen and one or more antennas positioned in the second lumen.

### Example 34

The UMC of any of examples 12-33, wherein that further includes a second lumen that includes one or more of: one or more sensors, and one or more antennas.

### Example 35

The UMC of example 33 or 34, wherein the second lumen has a length and includes one antenna that is at least half the length of the lumen.

### Example 36

The UMC of example 33 or 35, wherein the second lumen has a length and includes one antenna that extends at least 10% of the length.

### Example 37

The UMC of any of examples 12-33 that further includes an antenna that is in electrical contact with the one or more sensors.

### Example 38

The UMC of example 37, wherein the antenna is physically connected to the one or more sensors.

### Example 39

The UMC of example 12, wherein the at least one expansion structure is wing shaped.

### Example 40

The UMC of example 12, wherein the retainer portion comprises two expansion structures in the cavity and each expansion structure is configured to apply outward force on the outer cover.

### Example 41

The UMC of example 40, wherein each expansion structure is wing shaped.

### Example 42

The UMC of example 40, wherein each expansion structure extends outward and presses against an inner wall of the core.

### Example 43

The UMC of example 12, wherein each expansion structure has an intermediate section.

### Example 44

The UMC of example 12, wherein each expansion structure has a length that is at least 50% of the circumference of an inner wall of the outer cover.

### Example 45

The UMC of example 12, wherein each expansion structure has a length that is greater than 50% of the circumference of an inner wall of the outer cover.

### Example 46

The UMC of example 12, wherein each expansion structure has an intermediate section having a thickness that is 25% or more, or 30% or more, or 40% or more of a thickness of the outer cover.

### Example 47

The UMC of example 12 that further comprises an extendable flap on the distal end, wherein the extendable flap has a first, contracted position in which it is positioned against an outer wall of the tube, and a second, extended position in which it extends outward from the outer wall of the tube.

### Example 48

The UMC of example 47, wherein the extendable flap has a length that is at least 30%, or at least 40%, or at least 50%, or at least 60%, or at least 70% of an outer circumference of the tube.

### Example 49

The UMC of example 47, wherein the extendable flap has a thickness that is at least 50%, at least 60%, at least 70%, at least 80%, at least 90%, or at least 100% of a thickness of the tube wall.

### Example 50

The UMC of example 12 that further comprises a dual extendable flap on the distal end, wherein the dual extendable flap has a first flap, a second, flap, a first, contracted position in which the first flap and second flap are each positioned against an outer wall of the tube, and a second, extended position in which the first flap and second flap are each extend outward from the outer wall of the tube.

### Example 51

The UMC of example 12 that has a second wall of flexible material that defines a second lumen.

### Example 52

The UMC of example 51, wherein the lumen is configured to receive fluid.

### Example 53

The UMC of example 51 or 52, wherein the second wall is configured to expand.

### Example 54

The UMC of example 53, wherein the second wall can expand to 2-3 times its initial size.

### Example 55

The UMC of example 53, wherein the second wall can expand to 2-5 times its initial size.

Having thus described some embodiments of the invention, other variations and embodiments that do not depart from the spirit of the invention will become apparent to those skilled in the art. The scope of the present invention is thus not limited to any particular embodiment, but is instead set forth in the appended claims and the legal equivalents thereof. Unless expressly stated in the written description or claims, the steps of any method recited in the claims may be performed in any order capable of yielding the desired result. No language in the specification should be construed as indicating that any non-claimed limitation is included in a claim. The terms “a” and “an” expressly used in the context of describing the invention (especially in the context of the following claims) are to be construed to cover both the singular and the plural, unless otherwise indicated herein or clearly contradicted by context.

What is claimed is:

1. A method for measuring total urethral length utilizing a urethral measuring catheter (“UMC”) having a tube, a distal tip, a bulbar urethral anchor, and a bladder anchor, the method comprising the steps of:
(a) inserting the UMC through the urethra until the bladder anchor is positioned in the bladder;
(b) pulling back on the UMC until the bladder anchor is positioned against the bladder neck;
(c) marking or clamping the UMC at the meatus, or otherwise identifying the location of the UMC tube at the meatus; and
(d) removing the UMC from the body and measuring the distance from the location of the UMC tube as determined in step (c) at the meatus to a distal edge of the bladder anchor.

2. The method of claim 1 that further comprises the step of obtaining the measurement for the bladder neck to the external urinary sphincter by pushing forward on the UMC until resistance is felt from the urethral anchor pressing against the external urinary sphincter, measuring that position on the UMC, then pulling the UMC back to where resistance is felt by the bladder anchor pressing against the bladder sphincter, and subtracting that distance from the distance between the urethral anchor and the bladder anchor to obtain the measurement.

3. The method of claim 1, wherein the UMC has a tube and an outer, flexible tube external to the tube, and a space is defined between the tube and the outer, flexible tube.

4. The method of claim 1, wherein the bladder anchor is one or more extendable flaps.

5. The method of claim 1, wherein the urethral anchor is a retainer.

6. The method of claim 1, wherein the UMC further includes a tube having gradations or markings.

7. The method of claim 5, wherein the urethral anchor comprises and outer cover defining a cavity, and two expansion structures in the cavity, wherein each expansion structure is configured to apply outward force on the outer cover.

8. The method of claim 7, wherein each expansion structure is wing shaped.

9. The method of claim 7, wherein each expansion structure extends outward and presses against an inner wall of the outer cover.

10. The method of claim 8, wherein each expansion structure has an intermediate section.

11. The method of claim 8, wherein each expansion structure has a length that is at least 50% of the circumference of an inner wall of the outer cover.

12. The method of claim 8, wherein each expansion structure has a length that is greater than 50% of the circumference of an inner wall of the outer cover.

13. The method of claim 8, wherein each expansion structure has an intermediate section having a thickness that is 25% or more, or 30% or more, or 40% or more of a thickness of the outer cover.

14. The method of claim 1 that further comprises an extendable flap on the distal end, wherein the extendable flap has a first, contracted position in which it is positioned against an outer wall of a tube of the UMC, and a second, extended position in which it extends outward from the outer wall of the tube.

15. The method of claim 14 wherein the extendable flap has a length that is at least 30%, or at least 40%, or at least 50%, or at least 60%, or at least 70% of an outer circumference of the tube.

16. The method of claim 14, wherein the extendable flap has a thickness that is at least 50%, at least 60%, at least 70%, at least 80%, at least 90%, or at least 100% of a thickness of the tube wall.

17. The method of claim 1 that further comprises a dual extendable flap on the distal end of the UMC, wherein the dual extendable flap has a first flap, a second, flap, a first, contracted position in which the first flap and second flap are each positioned against an outer wall of the tube, and a second, extended position in which the first flap and second flap are each extend outward from an outer wall of the tube.

18. The method of claim 3, wherein the outer, flexible tube is configured to expand.

19. The method of claim 18, wherein the outer, flexible tube can expand to 2-5 times its initial size.

20. The method of claim 3 that further comprises the steps of (a) inserting fluid into the space and expanding the outer, flexible tube until the outer, flexible tube is in contact with a body part, and (b) measuring the diameter of the outer, flexible tube.
